(19) United States
(12) Patent Application Publication
(10) Pub. No.: US 2007/0184034 A1
(43) Pub. Date: Aug. 9, 2007

(54) Combination Pressure Therapy for Treatment of Hypertension, Blood Production, and Stem Cell Therapy

(75) Inventor: Carl Linton, Temecula, CA (US)

Correspondence Address: WILSON SONSINI GOODRICH & ROSATI, 650 PAGE MILL ROAD, PALO ALTO, CA, 94304-1050, US

(73) Assignee: CVAC SYSTEMS, INC., Temecula, CA (US)

(21) Appl. No.: 11/672,933

(22) Filed: Feb. 8, 2007

Related U.S. Application Data
(60) Provisional application No. 60/771,848, filed on Feb. 8, 2006.
(60) Provisional application No. 60/772,647, filed on Feb. 10, 2006.
(60) Provisional application No. 60/773,460, filed on Feb. 15, 2006.
(60) Provisional application No. 60/773,585, filed on Feb. 15, 2006.
(60) Provisional application No. 60/774,441, filed on Feb. 17, 2006.
(60) Provisional application No. 60/775,917, filed on Feb. 22, 2006.
(60) Provisional application No. 60/775,521, filed on Feb. 21, 2006.
(60) Provisional application No. 60/745,723, filed on Apr. 26, 2006.
(60) Provisional application No. 60/824,890, filed on Sep. 7, 2006.
(60) Provisional application No. 60/822,375, filed on Aug. 14, 2006.
(60) Provisional application No. 60/826,061, filed on Sep. 18, 2006.
(60) Provisional application No. 60/826,068, filed on Sep. 18, 2006.
(60) Provisional application No. 60/743,470, filed on Mar. 13, 2006.
(60) Provisional application No. 60/745,721, filed on Apr. 26, 2006.

(51) Int. Cl. A61K 38/17; A61K 35/14; A61B 5/00

(52) U.S. Cl. 424/93.7; 514/12; 600/300

(57) ABSTRACT

Methods for administering pressure changes to a user for the treatment and prevention of diseases and conditions are disclosed herein. Methods of administering Cyclic Variations in Altitude Conditioning Sessions (CVAC Session(s)) for the treatment of hypertension, blood production, and stem cell therapy are disclosed herein.

### CROSS-REFERENCE

This application claims the benefit of U.S. Provisional Application No. 60/771,848, filed Feb. 8, 2006, U.S Provisional Application No. 60/772,647, filed Feb. 10, 2006, U.S. Provisional Application No. 60/773,460, filed Feb. 15, 2006, U.S. Provisional Application No. 60/773,585, filed Feb. 15, 2006, U.S. Provisional Application No. 60/774,441, filed Feb. 17, 2006, U.S. Provisional Application No. 60/775,917, filed Feb. 22, 2006, U.S. Provisional Application No. 60/775,521, filed Feb. 21, 2006, U.S. Provisional Application No. 60/743,470, filed Mar. 13, 2006, U.S. Provisional Application No. 60/745,721, filed Apr. 26, 2006, U.S. Provisional Application No. 60/745,723, filed Apr. 26, 2006, U.S. Provisional Application No. 60/824,890, filed Sep. 7, 2006, U.S. Provisional Application No. 60/822,375, filed Aug. 14, 2006, U.S. Provisional Application No. 60/826,061, filed Sep. 18, 2006, and U.S. Provisional Application No. 60/826,068, filed Sep. 18, 2006, which applications are incorporated herein by reference.

### FIELD OF THE INVENTION

The invention relates to the use of air pressure therapy for the treatment and prevention of diseases and conditions that benefit from hypoxic conditioning.

### BACKGROUND OF THE INVENTION

Hypertension, commonly known as high blood pressure, is a source of multiple health problems and often precedes more significant health problems such as coronary disease, heart attacks, and strokes. Hypertension is thought to occur when the blood pressure inside the large arteries is too high. Hypertension affects roughly 50 million people in the United States alone and becomes more prevalent in older populations. Most cases of hypertension are of unknown etiology, but genetics is thought to play a role as hypertension can be inherited and manifests differently across ethnic and racial boundaries. Environment also plays a very important role in hypertension as do body weight and physical fitness. Additional factors related to the incidence and progression of hypertension include diet as well as a variety of medications with side effects known to increase blood pressure.

Other less common causes of hypertension include disorders of the kidneys or endocrine glands, and it has been called “the silent killer” in certain cases because it has no specific symptoms and yet can lead to death. People with untreated hypertension are much more likely to die from or be disabled by cardiovascular complications such as strokes, heart attacks, heart failure, arrhythmia, and kidney failure, as compared to people who have normal blood pressure. Current treatments for hypertension include lifestyle changes (diet, exercise, nonsmoking, etc.) as well as drug therapy. The major classes of medications currently used to treat hypertension include adrenergic neuron antagonists (which are peripherally acting), alpha adrenergic agonists (which are centrally acting), alpha adrenergic blockers, alpha & beta blockers, angiotensin II receptor blockers, angiotensin converting enzyme (ACE) inhibitors, beta adrenergic blockers, calcium channel blockers, Thiazide and related diuretics, and vasodilators, which act by direct relaxation of vascular smooth muscles. However, these known treatment regimens must be constantly monitored and adjusted, and most pharmaceutical treatments regimens are life-long.

Blood donation saves millions of lives every year by providing a blood source for patients in need of additional blood. Blood is lost during injuries, surgeries, births, and blood diseases, and medical facilities rely upon donated blood supplies in order to provide medical services. Maintaining blood supplies requires a steady supply of blood donors, and increased demand due to catastrophe and other problems (contaminated blood supply, failure of blood bank facilities, etc.) has strained blood supplies worldwide. Use of autologous blood has several advantages over generally donated blood. Such advantages include a guaranteed match of blood type, a reduced risk of infectious disease from contaminated blood, and no risk of allergic reaction. Autologous donation allows for a known volume of a donor's blood to be immediately accessible to the donor within the medical service organization where it was donated should the need arise. However, the same limitations on donation apply in autologous donation as with generally donated blood, and the restrictions on blood donation due to hemoglobin concentrations are especially detrimental for autologous donation by children awaiting major surgeries including open-heart surgery. [Sonzogni V, et al., Erythropoietin therapy and preoperative autologous blood donation in children undergoing open heart surgery, Brit. J. Anaesth., 87(3):429-34 (2001)] Considering the volume and frequency limitations on donation in addition to the low rate of donation in the population, blood banks are constantly searching for ways to improve the quality and quantity of their blood supply that do not require great expense, the use of pharmaceuticals or other agents that could scare donors away, or introduce further variables into the blood banking system.

In the early 1990's, researchers and the public began to focus on stem cells and their potential use for treatment of diseases. The identification of such a cell with the potential and ability to differentiate into any cell type present in an organism initially garnered interest in the treatment of autoimmune diseases and cancer due to the immediate correlation with hematopoiesis and suitability for genetic modification of a pluripotent precursor, but has since expanded into nearly all areas of human disease. In addition to bone marrow restoration treatments for cancers, such as leukemia, as well as autoimmune diseases, stem cell therapies are also under consideration for treatments including repair of organ tissues following disease on injury. These proposed stem cell therapies involve the administration of primary stem cells and/or modified stem cells to a specific tissue site in an organism. Notable areas of application include diabetes, hepatic disease, spinal cord regeneration, bone regeneration, ocular regeneration, and cardiac repair. [See e.g., Rajgobal, L, Stem Cell Therapy—A Panacea for all Ills?, J. Postgrad. Med. 51:161-163 (2005)].

Generally, stem cell therapies are limited by the supply of autologous stem cells. Initial efforts primarily utilized bone marrow aspiration techniques to harvest autologous stem cells (stem cells from one's own body) and heterologous stem cells (stem cells from a source other than one's own body). More recently, stem cells are preferably collected from a patient through a process called mobilization. Mobilization is achieved with the use of cytotoxic drugs and/or growth factors which are administered in very high dosages. Stem cell engraftment has a low rate of success, and many of the stem cells from the mobilization do not successfully implant despite the volume of cells administered, thus lengthening the recovery period as well as significantly increasing the costs associated with the procedure. [Joshi, S S., Miller, K., Jackson, J. D., Warkentin, P., and Kessinger, A., Immunological properties of mononuclear cells from blood stem cell harvests following mobilization with erythropoietin+G-CSF in cancer patients, Cytotherapy 2(1):15-24 (2002)].

Treatment Options and Needs

Current methods for prevention, treatment, and amelioration of hypertension are few. Similarly, known methods of improving erythropoiesis are few and require the administration of pharmaceuticals or other factors such as EPO to stimulate red blood cell production and increase blood volume. Known methods of stem cell mobilization, engraftment and recovery also primarily require the administration of pharmaceuticals and methods for improving efficiency of, and recovery from, stem cell transplantation are few. As stated, pharmaceutical intervention is the remedy of choice for all of the aforementioned indications. However, these pharmaceutical options are rarely used for general blood donation purposes, primarily due to cost. Additionally, regular and increased administration of exogenous hormones and molecules such as EPO can have detrimental results such as a decrease in oxygen carrying capacity of the blood due to extreme increases in hematocrit and increases in blood viscosity. Additionally, stem cell mobilization methods also require the use of large doses of toxic pharmaceuticals and growth factors. Thus there is a need for therapies which improve the treatment of the aforementioned indications. Further there is a need for additional therapies to work simultaneously or in concert with traditional methods for treating hypertension, improving erythropoiesis, and facilitating stem cell therapy. There is also a need for therapies without the potential negative side-effects of pharmaceutical and growth factor regimens. Alternatively, there is a need for such therapies that could lessen the negative side-effects of pharmaceutical and growth factor regimens by altering such regimens, that could work beneficially with pharmaceutical and growth factor regimens, or that could work synergistically when used in combination with pharmaceutical and growth factor regimens.

### SUMMARY OF THE INVENTION

The present invention provides for a method of administering pressure changes to a user for the purpose of treating hypertension, increasing blood and/or blood cell production (erythropoiesis), or facilitating stem cell therapy in the user. In an aspect of the invention, at least one CVAC session is administered to treat hypertension. In an embodiment of the invention, at least one CVAC session is administered to ameliorate the effects hypertension. In another embodiment, at least one CVAC session is administered to prevent hypertension. In another aspect of the invention, at least one CVAC session is administered for the stimulation of erythropoiesis. In yet another aspect of the invention, Cyclic Variations in Altitude Conditioning Sessions (CVAC sessions) are administered for the improvement of stem cell therapy. In an embodiment of the invention, at least one CVAC session is administered to improve stem cell mobilization. Another embodiment of the invention is the administration of at least one CVAC session for the improvement of stem cell engraftment. A further embodiment of the invention is the administration of at least one CVAC session for the improvement of recovery following stem cell therapies. In the aforementioned aspects and embodiments, multiple CVAC sessions may be administered. In the aforementioned aspects and embodiments, at least one CVAC session is administered in combination with alternative and/or standard therapies and methodologies, and/or in defmed intervals or at random occurrences. The effect of such administration is to prevent, treat, or ameliorate hypertension, to improve erythropoiesis, and to improve stem cell mobilization, stem cell engraftment, and/or stem cell transplantation recovery.

A CVAC session consists of a set of targets which are pressures found in the natural atmosphere. A CVAC session includes start and end points and more than one target executed between the start and end points. These targets are delivered in a precise order, and are executed in a variety of patterns including, but not limited to, cyclic, repeating or linear variations or any combination thereof. The starting points and ending points in any CVAC session are preferably the ambient pressure at the delivery site. The targets inherent in any CVAC session are connected or joined together by defined transitions. These transitions are either rises in pressure or falls in pressure, or a combination of the two. Additional targets which modulate time, temperature, or humidity are also run concurrently, sequentially, or at other intervals with the pressure targets when such additional targets and conditions are desired.

In an additional embodiment, including the aforementioned embodiments and aspects, the targets of the CVAC sessions include pressure, temperature, time, and humidity parameters. Parameters of targets and sessions can be customized to individual needs. In yet another embodiment of the invention, including the aforementioned embodiments and aspects, CVAC sessions are administered in combination with pharmaceutical regimens for the treatment, prevention, and amelioration of hypertension, improvement in blood production, and/or improvement of stem cell mobilization, stem cell engraftment, and recovery following stem cell therapy. Further embodiments, including the aforementioned embodiments and aspects, include administration of CVAC sessions in combination with alternative therapies and non-pharmaceutical therapies for the treatment, prevention, and amelioration of hypertension, improved erythropoiesis, and improvement of stem cell mobilization, stem cell engraftment, and recovery following stem cell therapy.

### BRIEF DESCRIPTION OF THE DRAWINGS

FIG. 1A depicts a graphed profile of the various pressures applied over time during an exemplary CVAC session. The Y-axis represents atmospheric pressure levels and the X-axis represents time. The varying pressures, as indicated by the changes in values on the Y-axis, were applied for various lengths of time, as indicated by changes values on the X-axis. The exemplary CVAC session depicted in FIG. 1A was 20 minutes in length.

FIG. 1B depicts a different graphed profile of the pressures applied over time during another exemplary CVAC session. The Y-axis again represents atmospheric pressure levels and the X-axis represents time. Different pressures were again applied, as indicated by changes in value on the Y-axis, for various lengths of time, as indicated by the changes in values on the X-axis. This exemplary CVAC session was also 20 minutes in length.

### DETAILED DESCRIPTION OF THE INVENTION

A Pressure Vessel Unit (PVU) is a system for facilitating pressure changes accurately and quickly in the environment surrounding a user. A PVU can provide both reduced and increased atmospheric pressures. An example of a unique PVU and associated methods for controlling the pressure within such a PVU are described in U.S. Patent Publication number 2005/0056279 A1 and incorporated herein by reference. A variety of PVUs may be used in conjunction with the methods disclosed herein, including but not limited to those described in the U.S. Patent Publication number 2005/0056279, such as variable or fixed pressure and temperature hypobaric units. Other pressure units or chambers will be known to those of skill in the art and can be adapted for use with the disclosed methodologies.

Methodology of the Cyclic Variations in Altitude Conditioning (CVAC) Program:

The methodology of the present invention encompasses a set of pressure targets with defined transitions. Additional targets can be included such as temperature or humidity, and these targets can be implemented concurrently, prior to, or subsequent to the pressure targets. The permutations of targets are customizable to the individual and condition to be treated. Alternatively, a user may also modulate the parameters of a CVAC session, in certain embodiments from within the unit, thus providing for real-time user feedback and alterations. As used in reference to parameter of a CVAC session, modulation includes any changes, positive and negative, made to the parameters of the CVAC session. The parameters are described herein. Some of the terms relating to this methodology are defined below for a better understanding of the methodology as used in the context of the present invention.

A CVAC Program:

Every user will respond in a unique manner to changes in air pressure, temperature and oxygen levels that occur during cyclic variations in altitude conditioning. This necessitates a customized approach to delivering a highly effective and efficacious Program to each user The Program consists of a set of sessions, which are administered to the user as a serial round or cycle. This means that a user may have a session that they start and repeat a given number of times and then proceed to the next scheduled session which will be repeated a given number of times. A program may contain a set of one or more sessions, each of which preferably has a repetition schedule. The sessions are preferably delivered in a scheduled order, which repeats itself like a loop such that the user is administered one session at a time for a specified number of times. The user is then administered the next scheduled session a specified number of times. This process is preferably repeated until the user is administered the last element of the scheduled sessions set. When the requisite repetitions have been accomplished, preferably the process repeats itself beginning at the first element of the scheduled sessions set. A session or groups of sessions may be repeated multiple times before changing to a subsequent session or group of sessions, however, sessions may also be administered as few as one time before beginning the next session in the sequence. Subsequent sessions can contain targets that are identical to the previous session, or they can implement new permutations of desired targets. The combination of sessions and targets within sessions is customizable based on the desired physiological outcome and assessment of the user. This comprises a Cyclic Variations in Altitude Conditioning (CVAC) Program.

A CVAC Session:

A CVAC Session comprises of a set of targets which are multiple atmospheric pressures, and a CVAC session includes start and end points, and more than one target which is executed between the start and end points. These targets are delivered in a precise order that may vary and are executed in a variety of patterns including, but not limited to, cyclic, repeating, and/or linear variations. When a target is executed as contemplated herein, executed includes a change in pressure from one pressure value to another pressure value within a CVAC device as also described herein. The methodologies described herein are superior to previously described static hypobaric pressure therapies in multiple ways, which can include reduced time frames of application and unique variations and combinations of atmospheric pressures. Furthermore, CVAC sessions can also provide beneficial effects via the vas-pneumatic properties associated with the application of such sessions. The starting points and ending points in any CVAC Session are preferably the ambient pressure at the delivery site. The targets inherent in any CVAC Session are connected or joined together by defined transitions. These transitions are either increases in pressure (descent) or decreases in pressure (ascent), or a combination of the two. The nature of any transition may be characterized by the function of “delta P/T” (change in pressure over time). Transitions may be linear or produce a waveform. Preferably, all transitions produce a waveform. The most desirable waveforms are Sine, Trapezoidal and Square. Additional targets which modulate time, temperature, or humidity and combinations thereof are also run concurrently, sequentially, or at other intervals with the pressure targets when such additional targets and conditions are desired. The entire collection of targets and transitions are preferably delivered in a twenty minute CVAC Session, although the time of each session may vary in accordance with the desired outcome of the administration of the CVAC Sessions. For example, CVAC sessions may be administered over minute increments such as 5, 10, 15, 16, 17, 18, 19, 20, 25, 30 minutes or more. The length of each CVAC Session is customizable for each user.

A Set-Up Session:

The Set-Up Session may also be considered a Program. It is a single Session designed to prepare a new user for the more aggressive maneuvers or transitions encountered in the subsequent Sessions that the user will undergo. The Set-Up Session accounts for all ages and sizes and conditions, and assumes a minimal gradient per step exercise that allows the ear structures to be more pliant and to allow for more comfortable equalization of pressure in the ear structures. The purpose of the Set-Up Session is to prepare a new user for their custom Program based upon the group into which they have been placed. The function of the Set-Up Session is to qualify a user as being capable of adapting to multiple pressure changes in a given Session with acceptable or no discomfort. Set-Up session transitions may be linear or produce a waveform. Preferably, all transitions are linear. This is accomplished by instituting a gradient scale increase in pressure targets from very slight to larger increments with slow transitions increasing until a maximum transition from the widest difference in pressure targets is accomplished with no discomfort. The structure of a preferred Set-Up Session is as follows: as with any Session, the starting point and ending point is preferably at ambient pressure. A target equivalent to 1000ft above ambient is accomplished via a smooth linear transit. A second target equivalent to 500ft less than the first target is accomplished via a slow to moderate transit. These two steps are repeated until the user returns a “continue” or “pass” reply via an on-board interface. When the user has indicated that they are prepared to continue, the initial target (1000ft) is increased by a factor of 500ft, making it 1500 ft. The secondary target (500ft less than the first target) remains the same throughout the session until the exit stage is reached. Each time the user indicates that they are ready to increase their gradient, the target is increased by a factor of 500ft. At this time, the transits remain the same but the option of increasing gradient (shorter time factor) in the transits is available. A user preferably has the option of resuming a lower gradient if desired. There can be an appropriate icon or pad that allows for this option on the on-board interface display screen. Preferably, the Set-Up Session lasts no longer than 20 minutes. A Set-Up session typically runs for twenty minutes maximum and executes a final descent to ambient atmospheric pressure upon beginning the last transit. The Set-Up Session is a new user's Program until the user is able to fully complete the Set-Up Session (that is to continue the targets and transits to the highest gradient) with no interrupts or aborts. When administering CVAC sessions for medical treatment, Set-Up Sessions may be customized to suit the requirements of their medical condition. The determination of the appropriate Set-Up Session can be made with guidance from or consultation with a user's qualified health professional, such as a treating physician.

The Interrupt:

During any phase in a Session wherein a user desires to stop the Session at that point for a short time, they may do so by activating an icon or other appropriate device on the on-board interface touch screen or control pad. This will hold the Session at the stage of interruption for a predetermined time period, such as a minute, at which time the Session will continue automatically. Preferably, a Session may be interrupted three times after which a staged descent will occur and the user will be required to exit the pressure vessel. The user's file will be flagged and the user will be placed back on the Set-Up Sessions until they can satisfactorily complete it. A warning or reminder may be displayed on the screen each time an interrupt is used that informs the user of how many times interrupt has been used and the consequences of further use. During any session, be it a Set-Up session or other type of session, a staged descent is also available if the user develops ear or sinus discomfort or wishes to terminate the session for any reason. A staged descent is characterized by slow, 1000 ft sine wave descent transits with re-ascensions of 500ft at each step. The descents can be of greater or lesser transits but the ratio is usually about 1.5:1. At any time during the staged descent, the user can interrupt the descent and hold a given level or resume a previous level until comfort is achieved. The user may also re-ascend at their option if the staged descent is too aggressive. Any re-ascension is done in stages as described above. The user can indicate a “continue” on the descent and the staging will resume. This stepping continues until ambient pressure is reached whereupon the canopy opens such that the user can exit the pressure vessel.

The Abort:

When a user wishes to end a Session immediately and quickly exit the pressure vessel, the abort function can be activated. Touching the “abort” icon on the on-board interface touch pad/screen enables this option. A secondary prompt is activated acknowledging the command and asking the user if they are sure they want to abort. The user indicates their commitment to the command by pressing “continue” or “yes”. The Program is aborted and a linear moderate descent is accomplished to ambient pressure whereupon the canopy opens and the user exits. The user's file is flagged. The next time the user comes in for their Session, the user is asked whether the abort was caused by discomfort. If yes, the user is placed back on the Set-Up Session Program. If no, the user is asked if they wish to resume their regularly scheduled Session. The client is given the option of resuming their regularly scheduled Session or returning to the Set-Up Session.

Program and Target Criteria, Including Medically Significant Criteria:

Preferably, a user is categorized into a group of users having similar body-types with similar characteristics based upon answers to a questionnaire. The information from the questionnaire guides the construction of custom CVAC programs for each individual. When administering CVAC programs for treatment of hypertension, improved blood production, or stem cell therapy, the medical status of the user can also be used to determine appropriate pressures and additional parameters (such as duration, temperature, or humidity) of the targets. Custom session targets may be administered based upon the medical condition and therapy desired. The acceptable and appropriate target parameters may be obtained through consultation with the user's physician or other appropriate health-care provider prior to designing session targets and administering a CVAC session. However the known contraindications of CVAC are similar to those of commercial air travel, allowing for a broad range of application.

Hypoxic Conditioning:

Initial understanding in the art about the effects of hypoxia focused on increased oxygenation of the blood via increased production of red blood cells mediated by increases in EPO production. While increases in EPO production are believed to increase red blood cell production, its effects are not limited to this activity. Molecules such as HIF, induced by hypoxia, regulate EPO production in addition to a variety of other activities including metabolism, angiogenesis, and vascular tone—the stimulation of which may all play a role in protecting tissue from subsequent hypoxic damage. This protection may occur prophylactically, post-ischemic or traumatic events as well as facilitating stem cell mobilization and red blood cell production. [Eckardt K.U., Kurtz, A., Regulation of erythropoietin production, Eur. J. Clin. Invest., 35(Supp. 3):13-19, (2005)]. Attempts to improve blood donation volume and frequency have focused on the administration of erythropoietin. Erythropoietin is known to induce red blood cell production, thus increasing red blood cell volume in the patient. [Kirsh KA, et al., Erythropoietin as a volume-regulating hormone: an integrated view. Semin. Nephrol., 25(6):388-91 (2005)] Recent research demonstrated the dramatic increase in red blood cell volume in children following the administration of erythropoietin. This increase allowed for autologous donation by the children of the study prior to undergoing open-heart surgery. The increase in red blood cell volume prevented drops in red blood cell volumes typically associated with blood donation, especially in children. The maintenance of stable red blood cell counts despite repeated donations in a 20 day period allowed for autologous donation of sufficient blood volumes in anticipation of each child's surgery as well as maintained sufficient blood counts to allow for subsequent surgery. [Sonzogni V, et al., Erythropoietin therapy and preoperative autologous blood donation in children undergoing open heart surgery, Brit. J. Anaesth., 87(3):429-34 (2001)]. Additional studies have also demonstrated the effectiveness of erythropoietin in improving red blood cell volumes, donation volumes, and ability to donate multiple times. [Goodnough L T, et al., Preoperative red cell production in patients undergoing aggressive autologous blood phlebotomy with and without erythropoietin therapy, Transfusion, 32(5):441-5 (1992); Biesma D H, et al., The efficacy of subcutaneous recombinant human erythropoietin in the correction of phlebotomy-induced anemia in autologous blood donors, Transfusion 33(10):825-9 (1993)]

In addition to EPO administration, therapies such as oxygen deprivation at static air pressures and static blocks of time are known to provide some beneficial effects for increasing red blood cell production, oxygenation of the blood and hematocrit. [Heinicke K, et al., Long-term exposure to intermittent hypoxia results in increased hemoglobin mass, reduced plasma volume, and elevated erythropoietin plasma levels in man, Eur. J. Appl. Physiol., 88(6):535-43 (2003)]. While oxygen deprivation of the body or specific tissues can cause tissue damage, and even death, controlled deprivation of oxygen to the body or specific tissues or a combination thereof may be beneficial when imposed for specific periods of time under particular conditions. Static hypoxic conditioning may be provided by decreased oxygen levels in the atmosphere or by a reduction in atmospheric pressure (hypobaric conditions), thus reducing the availability of oxygen for efficient respiration.

Attempts to improve stem cell mobilization, engraftment, and post-transplantation recovery have focused on the administration of erythropoietin. Erythropoietin (EPO) is known to induce red blood cell production, thus increasing red blood cell volume in the patient. [Kirsh K A, et al., Erythropoietin as a volume-regulating hormone: an integrated view. Semin. Nephrol., 25(6):388-91 (2005)] Typical mobilization protocols utilize the cytokine granulocyte colony stimulating factor (G-CSF). However, the addition of EPO is also known to boost hematopoietic precursor cells (stem cells) as well as immune effector cells, thus improving the collection during mobilization and increasing the percentage of cells for successful engraftment. [Joshi, S S., Miller, K., Jackson, J. D., Warkentin, P., and Kessinger, A., Immunological properties of mononuclear cells from blood stem cell harvests following mobilization with erythropoietin+G-CSF in cancer patients, Cytotherapy 2(1):15-24 (2002)]. A final mobilization factor is the cytokine vascular endothelial growth factor (VEGF). In additional to stimulating angiogenesis, VEGF has been linked with increased mobilization of stem cells from the bone marrow, thus providing another factor for improving pre-transplantation mobilization.

Following transplantation, EPO may also play a role in improving reconstitution of the patient' hematopoietic system. The combination of EPO+G-CSF can accelerate successful engraftment following stem cell transplantation. [Id.; Dempke, W. and Schmoll, H. J., Possible new indications for erythropoietin therapy, Med.Klin. (Munich), 96(8):467-74 (2001)]. The improvement in successful engraftment is directly correlated with an improvement in reconstitution of the blood in the patient. Administration of EPO is known to improve the recovery time following stem cell transplantation, likewise by improving the reconstitution of the peripheral blood red-blood cell numbers and by reducing the amount of transfusions needed during recovery. Decreased recovery time also reduces the window for complicating opportunistic infections and other post-transplantation care, and will reduce costs and improve recovery. [Ivanov, V., Fuacher, C., Mohty, M., Bilger, K., Ladaique, P., Sainty, D., Arnoulet, C., Chabannon, C., Vey, N., Camerlo, J., Bouabdallah, R., Viens, P., Maraninchi, D., Bardou, V. J., Estemi, B., and Blaise, D., Early administration of recombinant erythropoietin improves hemoglobin recovery after reduced intensity conditioned allogeneic stem cell transplantation, Bone Marrow Transplant., 36(10):901-06 (2005); Vanstraelen, G., Baron F., Frere, P., Hafraoui, K., Fillet, G., and Beguin, Y. Efficacy of recombinant human erythropoietin therapy started one month after autologous peripheral blood stem cell transplantation, Haematologica, 90(9):1269-70 (2005)].

Moderate static hypoxic preconditioning is known to provide protection from tissue and cellular damage via tolerance. When the environmental oxygen levels are reduced (hypoxia), downstream effects include protection from damage due to subsequent hypoxia. [Sharp, F., et al., Hypoxic Preconditioning Protects against Ischermic Brain Injury, NeuroRx: J. Am. Soc. Exp. Neuro., Vol.1: 26-25 (2004)]. This tolerance is not yet completely understood, but it has been linked to various cellular mechanisms and molecules, including, but not limited to, molecules such as erythropoietin (EPO), hypoxia-inducible factor (HIF), Tumor Necrosis Factor (TNF), glycogen, lactate, and others. [Sharp, F., et al., Hypoxic Preconditioning Protects against Ischemic Brain Injury, NeuroRx: J. Am. Soc. Exp. Neuro., Vol. 1:26-25 (2004)]. Additionally, beneficial static hypoxic conditioning is not purely additive. Administration of sequential sessions can have detrimental effects. Oxygen concentrations that are too low result in detrimental effects to the tissues as well as the entire body. Similarly, hypoxia conditioning of longer durations can have detrimental effects in addition to providing some desired beneficial effects. [Sharp, F., et al., Hypoxic Preconditioning Protects against Ischemic Brain Injury, NeuroRx: J. Am. Soc. Exp. Neuro., Vol. 1:26-25 (2004)]. Furthermore, prior hypoxic conditioning studies utilized static pressures over lengthy time-frames. In contrast to the aforementioned static hypoxic conditioning known in the art, CVAC sessions utilize multiple variations in altitudes and variable, time-frames of application. The combination of varying pressures over varying time frames, including rapid changes over varying time-frames, produces multiple beneficial effects associated with hypoxic condition, stimulates additional beneficial effects, and does not result in the detrimental effects seen with static hypoxic conditioning. Similarly, the duration of CVAC sessions, while not limited, are typically much shorter than the long blocks of time currently used for static hypobaric conditioning. Thus, the use of unique CVAC sessions for the production of beneficial hypoxic effects provides a novel and superior alternative to the current methods of static hypoxic conditioning as described above.

Methods of Treatment:

CVAC sessions for hypertension, blood production, and stem cell therapy, including but not limited to uses to aid in stem cell mobilization, stem cell engraftment, and recovery following transplantation, are administered preferably for at least 10 minutes, and more preferably at least 20 minutes, with variable frequency. CVAC sessions are administered preferably for at least 10 minutes, and more preferably at least 20 minutes, with variable frequency. Additional administration periods may include, but are not limited to, about 10 minutes, about 20 minutes, about 30 minutes, about 40 minutes, about 60 minutes, between 10 and 20 minutes, between 20 and 30 minutes, between 30 and 60 minutes, and between 60 and 120 minutes. Frequencies of sessions or series of sessions may include, but are not limited to, daily, monthly, or when medically indicated or prescribed. The frequency and duration of the sessions can be altered to suit the medical condition to be treated, and CVAC sessions may be administered as single sessions, or as a series of sessions, preferably with a Set-Up Session as described herein. For example, the frequency of sessions or series of sessions can be administered 3 times a week for 8 weeks, 4 times a week for 8 weeks, 5 times a week for 8 weeks, or 6 times a week for 8 weeks. Additional frequencies can be easily created for each individual user. Similarly, the targets in the sessions can also be altered or adjusted to suit the individual and medical condition to be treated. The permutations of targets can be customized to the individual, and may again be identified with the help of any person skilled in the art, such as a treating physician. Furthermore, the variations may be administered in regular intervals and sequence, as described, or in random intervals and sequence. The variations in number, frequency, and duration of targets and sessions can be applied to all methods of treatment with CVAC described herein.

In one aspect of the present invention, administration of CVAC sessions prior to development of clinical hypertension or related hypertensive conditions can prophylactically treat and/or aid in the prevention of hypertension. In one embodiment, prophylactic administration of CVAC sessions can also prevent or reduce the tissue damage in subsequent hypertensive events. The ability of CVAC sessions to increase the blood flow, stimulate angiogenesis, modulate blood lipid patterns, and stimulate protective cellular responses conditions can condition tissues and vessels to prevent progression to a state of hypertension. As defined herein, treatment of hypertension includes administration of at least one CVAC session for the prevention of hypertension (ie: prior to diagnosis), administration of at least one CVAC session for treatment of hypertension, and administration of at least one CVAC session for the amelioration of hypertension.

Additionally, CVAC sessions are believed to act like a vaso-pneumatic pump on the user's body, thus stimulating flow of fluids in the body, including but not limited to, blood and lymphatic fluids. The negative and positive pressures imposed by the CVAC session affect the fluid flow or movement within a body, thus improving the delivery of beneficial nutrients, immune factors, blood, and oxygen while also improving the removal of harmful toxins, fluids, and damaged cells or tissues. The combination of the beneficial effects of CVAC sessions results in treatment of hypertension and related conditions.

In an additional aspect of the present invention, Cyclic Variations in Altitude Conditioning Program is used to treat users who wish to increase their production of blood or those who wish to shorten the recovery time required between blood extraction or withdrawal (commonly referred to as donation). CVAC is administered to increase the oxygenation of the blood, increase the number of red blood cells within a user, increase the production of HIF's, and/or stimulate other associated physiological processes affected by CVAC treatment such as fluid (lymph, blood, or other bodily fluids) movement. Treatment is administered through the use of one or more CVAC sessions. Such sessions may be user defined or custom-defined with input from the user's physician. CVAC sessions may be administered in advance of any surgeries or other treatment regimens to increase production and quality of blood for more efficient and frequent blood donation.

In yet another aspect of the present invention, Cyclic Variations in Altitude Conditioning Program is used to treat users who are in need of stem cell therapy. As defined herein, stem cell therapy includes mobilization, engraftment and recovery following a stem cell therapy. In one embodiment, CVAC is administered to mobilize stem cells into the blood. As used herein, mobilization includes mobilization of stem cells in any source (autologous, heterologous, etc.) In another embodiment, CVAC is administered to facilitate engraftment of stem cells in a user. In yet another embodiment, CVAC is administered to facilitate recovery following stem cell therapy. As used herein, a method to mobilize stem cells includes the administration of at least one CVAC session prior to or following a mobilization procedure. Furthermore, a method to mobilize stem cells also includes administration of at least one CVAC session at defined or random intervals. Similarly, methods to facilitate engraftment as disclosed herein also include, but are not limited to, the administration of at least one CVAC session prior to, during, or following an engraftment procedure, and these too can be administered at defined or random intervals. Finally, methods to facilitate recovery from stem cell therapies also include, but are not limited to, the administration of at least one CVAC session prior to, during, or following a stem cell procedure, and these too can be administered at defined or random intervals. CVAC sessions for stem cell therapies are administered to increase the oxygenation of the blood, increase the number of red blood cells within a user, increase the production of HIF's, and/or stimulate other associated physiological processes affected by CVAC treatment such as fluid (lymph, blood, or other bodily fluids) movement. Treatment is administered through the use of one or more CVAC sessions. Such sessions may also be user defined or custom-defined with input from the user's physician. CVAC sessions may be administered in advance of any surgeries or other treatment regimens to mobilize stem cells, preferably more productively and efficiently than standard therapies, engraft stem cells, preferably more efficiently than standard therapies, and facilitate recovery following stem cell therapies, preferably faster and more efficiently than standard therapies

Although not limited to a particular mechanism of action, it is believed that the ability of CVAC therapy to provide increased blood flow, increased red blood cell counts, angiogenic and protective cellular responses, EPO production, VEGF production, and HIF production can aid in treatment, prevention, and amelioration of hypertension, improve erythropoiesis, and modulate mobilization, engraftment, and recovery following stem cell therapies. Additionally, CVAC sessions are believed to act like a vaso-pneumatic pump on the user's body, thus stimulating flow of fluids in the body, including but not limited to blood and lymphatic fluids. The negative and positive pressures imposed by the CVAC session affect the fluid flow or movement within a body, thus improving the delivery of beneficial nutrients, immune factors, blood, and oxygen while also improving the removal of harmful toxins, fluids, and damaged cells or tissues. The combination of the beneficial effects of CVAC sessions results in prevention, treatment, and/or amelioration of hypertension. Similarly, the beneficial effects of CVAC sessions result in improved erythropoiesis. Finally, CVAC sessions also beneficially effect mobilization and engraftment of stem cells as well as modulation of the recovery time following stem cell therapy. Additionally, CVAC is not limited to application with stem cell transplantation of the bone marrow, and CVAC sessions may be administered in a similar manner to any type of stem cell therapy involving the mobilization, collection, and/or administration of stem cells.

Modulating, in the context of assessment of CVAC sessions, has multiple meanings. In the context of hypertension, modulation means reduction in blood pressure in the user. In the context of blood production, modulation means any changes that result in the increased numbers of red blood cells, hematocrit, or blood volume. Additionally, modulation in the context of improved erythropoiesis means any shortening of the time between successful blood extractions. Finally, modulation in the context of stem cell therapy means increases in stem cell mobilization, reduction in recovery time compared to standard therapies, less painful recovery compared to standard therapies, and/or more robust responses in physiological parameters compared to standard therapies.

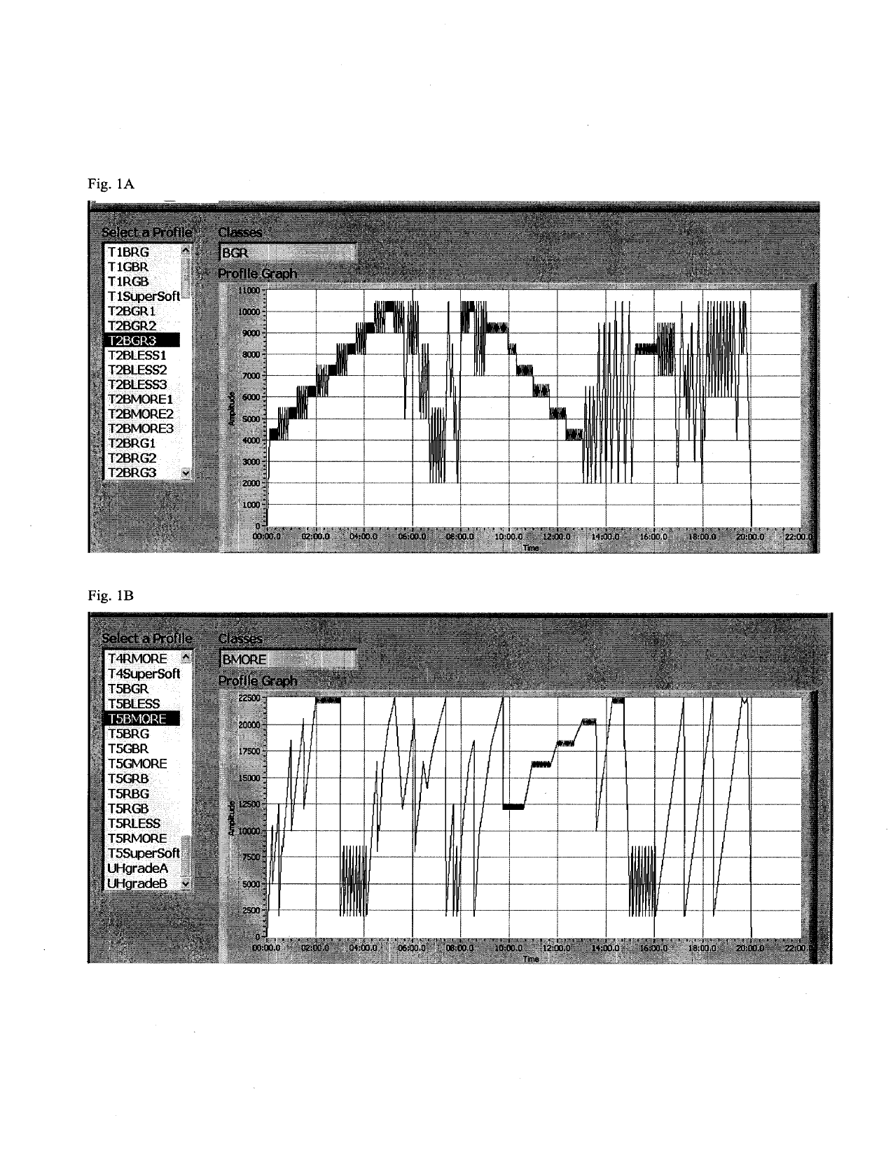

Specific examples of a CVAC session are shown graphically in FIGS. 1A and 1B. In both figures, the parameters of the program are shown as a line graph with axes that correspond to time (x-axis) and pressure change (y-axis).

CVAC sessions may also be used in combination with pharmaceutical and growth factor regimens or non-pharmaceutical therapies including but not limited to herbal supplements, vitamins, nutritional changes, and exercise regimens believed to assist in blood production. As described above, CVAC sessions of any combination or permutation can be administered prior to, concurrent with, or subsequent to administration of a pharmaceutical, pharmaceuticals, or non-pharmaceutical therapy. Myriad permutations of pharmaceutical therapies, non-pharmaceutical therapies, and CVAC session combinations are possible, and combinations appropriate for the type of medical condition and specific pharmaceutical may be identified with the help of any person skilled in the art, such as a treating physician.

Efficacy of Treatment

Hypertension

Efficacy of CVAC treatments for prevention and treatment of hypertension can be evaluated with a variety of imaging and assessment techniques known in the art. Imaging examples include methods such as magnetic resonance imaging (MRI) of the affected region such as blood vessels and/or the heart, invasive imaging through catheterization, or alternative non-invasive imaging methods. Additional assessment criteria known in the art include: blood pressure analysis, blood and/or plasma lipid profiling, hematocrit measurement, blood-gas analysis, extent of blood-perfusion of tissues, angiogenesis within tissues, erythropoietin production, VEGF production, modulation of HIF-1α and associated gene expression, extent of tissue necropsy following ischemic events, and assessment of cognitive abilities and/or motor skills following ischemic events.

By example only, when blood or plasma lipid levels are the physiological markers used to assess CVAC efficacy, modulation of blood or plasma lipid levels during or following one or more CVAC sessions is indicative of efficacious CVAC treatment for the treatment, amelioration, or prevention of hypertension. In one embodiment, an increase in HDL cholesterol is indicative of efficacious CVAC treatment. Conversely, a lack of change in the user's HDL cholesterol (or with any of the physiological markers described herein) does not necessarily indicate that the CVAC treatments are not achieving positive results. Similarly, when blood pressure analysis is the physiological marker used to assess CVAC efficacy, modulation of the blood pressure during or following one or more CVAC sessions is indicative of efficacious CVAC treatment. When blood-perfusion of the tissues is the physiological marker used to assess CVAC efficacy, increases in blood volumes and/or blood exchange within tissues during or following one or more CVAC sessions are indicative of the efficacious CVAC treatment. Angiogenesis within affected tissues can also be a physiological marker used to assess CVAC efficacy. Modulation of vessel development within the affected tissues during or following one or more CVAC sessions is indicative of efficacious CVAC treatments. Additionally, initiation or modulation of VEGF expression within affected tissues during or following one or more CVAC sessions is also indicative of efficacious CVAC treatment. Modulation of HIF-1α following one or more CVAC sessions is also a physiological marker used to assess the efficacy of CVAC treatments. In one embodiment of the present invention, increases in the expression of HIF-1α indicate efficacious CVAC treatments. Extent of tissue necropsy is a further physiological marker used to assess CVAC efficacy. Additional criteria for assessing the treatment and prevention of ischemic damage or ischemic events will be known by those of skill in the art and can be employed to assess the beneficial effects of CVAC programs.

In one embodiment, a CVAC user's blood pressure is analyzed prior to initial use of CVAC, following one or more CVAC sessions, and/or following the completion of any given series of CVAC sessions. Blood pressure is taken prior to beginning the initial and/or each subsequent CVAC session therapy and again at designated time points following the administration of one or more CVAC sessions. Appropriate time points for measurements taken following the administration of one or more CVAC sessions include, but are not limited to, time points immediately following a one or more CVAC sessions, time points following the CVAC sessions sufficient to allow a user's physiological indicators or parameters to return to a normal or resting state, and/or any additional time points known to one of skill in the health or medical profession. [Pickering, T.G., et al., “Recommendations for Blood Pressure Measurement in Humans and Experimental Animals: Part 1: Blood Pressure Measurement in Humans: A statement for Professionals From the Subcommittee of Professional and Public Education of the American Heart Associate Council on High Blood Pressure Research” (2005) Hypertension 45: 142-161.; Kurtz, T. W. et al., “Recommendations for Blood Pressure Measurement in Humans and Experimental Animals: Part 2: Blood Pressure Measurement in Experimental Animals: A statement for Professionals From the Subcommittee of Professional and Public Education of the American Heart Associate Council on High Blood Pressure Research” (2005) Hypertension 45: 299-310.] A drop and/or slower increase over time in one or both systolic and diastolic pressures indicates efficacy due to the administration of one or more CVAC sessions. Blood pressure may be monitored beyond administration of one or more CVAC sessions to assess continued drops in blood pressure following administration of one or more CVAC sessions.

In another embodiment, blood pressure may be analyzed to assess the efficacy of CVAC sessions for prevention of hypertension. A user's blood pressure is monitored prior to administration of one or more CVAC sessions and then again subsequent to the administration of one or more CVAC sessions. The results are then compared to the blood pressure norms based upon studies to determine the clinically normal range of blood pressure from a population that has one or more known risk factors for developing hypertension. Such risk factors include, bur are not limited to, genetic predisposition, unhealthy body weight, a diet high in fats and/or sodium, a tobacco user, typical “high stress” jobs or work environments, and any other risk factors known and recognized by one of skill in the field of health and hypertension. A drop in a user's blood pressure relative to the control following administration of one or more CVAC sessions is indicative of efficacious CVAC treatment for the prevention of hypertension.

In a related embodiment, prevention of hypertension by monitoring blood pressure may also be assessed through comparison to a drop in blood pressure such that hypertension is less likely based on medically accepted hypertension diagnosis parameters. [Pickering, T.G., et al., “Recommendations for Blood Pressure Measurement in Humans and Experimental Animals: Part 1: Blood Pressure Measurement in Humans: A statement for Professionals From the Subcommittee of Professional and Public Education of the American Heart Associate Council on High Blood Pressure Research” (2005) Hypertension 45: 142-161.; Kurtz, T. W. et al., “Recommendations for Blood Pressure Measurement in Humans and Experimental Animals: Part 2: Blood Pressure Measurement in Experimental Animals: A statement for Professionals From the Subcommittee of Professional and Public Education of the American Heart Associate Council on High Blood Pressure Research” (2005) Hypertension 45: 299-310.] The diagnosis of hypertension depends upon a variety of factors including a blood pressure above an upper limit of normal. A lowering of a user's blood pressure from a measurement nearer to the upper limit of normal to a measurement further from said limit is indicative of CVAC session administration efficacy in preventing hypertension. Again, one of skill in the diagnosis, treatment, and prevention of hypertension such as a medical doctor can aid in this determination of efficacy and will know further means of assessing prevention of hypertension following administration of one or more CVAC sessions. The embodiments described herein for assessing CVAC efficacy in preventing hypertension are not limited to use with blood pressure analysis, and they may be applied to any of the aforementioned physiological markers for similar assessment.

Erythropoiesis

Efficacy of CVAC treatments for red blood cell production can be evaluated with a variety of imaging and assessment techniques known in the art. Assessment criteria known in the art include: hematocrit measurement, blood-gas analysis, extent of blood-perfusion of tissues, angiogenesis within tissues, erythropoietin production, and recovery of blood volume and red blood cell counts. Additional criteria for assessing the production of red blood cells will be known by those of skill in the art and can be employed to assess the beneficial effects of CVAC programs.

By example only, modulation of hematocrit is indicative of CVAC efficacy for red blood cell production. Conversely, a lack of change in the user's hematocrit (or with any of the physiological markers described herein) does not necessarily indicate that the CVAC treatments are not achieving positive results. Angiogenesis within affected tissues can also be a physiological marker used to assess CVAC efficacy. Modulation of vessel development within the tissues or body of a user during or following one or more CVAC sessions is indicative of efficacious CVAC treatments. Again, by example only, angiogenesis may be assessed by a variety of imaging and detection methods including dyes, MR, fluoroscopy, endoscopy, and other means known in the art. Additionally, initiation or modulation of VEGF expression within affected tissues during or following one or more CVAC sessions is also indicative of efficacious CVAC treatment. Modulation of erythropoietin production following one or more CVAC sessions is also a physiological marker used to assess the efficacy of CVAC treatments. In one embodiment of the present invention, increases in the expression of erythropoietin indicate efficacious CVAC treatments. Similarly, when blood-gas analysis is the physiological marker used to assess CVAC efficacy, modulation of the dissolved gasses in the blood during or following one or more CVAC sessions is indicative of efficacious CVAC treatment. Typical gasses monitored include oxygen, carbon dioxide, and nitrogen. However, any gas found within the blood may be monitored for assessment of CVAC efficacy. When blood-perfusion of the tissues is the physiological marker used to assess CVAC efficacy, increases in blood volumes or blood exchange and combinations thereof within tissues during or following one or more CVAC sessions are indicative of the efficacious CVAC treatment. Additional criteria for assessing the production of red blood cells will be known by those of skill in the art and can be employed to assess the beneficial effects of CVAC programs.

Stem Cell Therapy

Efficacy of CVAC treatments for mobilization of stem cells, engraftment of stem cells, and recovery following stem cell therapy can be evaluated with a variety of imaging and assessment techniques known in the art. Assessment criteria known in the art include, but are not limited to: assessment of EPO levels, assessment of VEGF levels, assessment of cytokine profiles, peripheral blood stem cell counts, peripheral blood immune effector cell counts, hematocrit measurement, blood-gas analysis, extent of blood-perfusion of tissues, angiogenesis within tissues, , and recovery of blood volume and red blood cell counts. Additional criteria for assessing the production of red blood cells will be known by those of skill in the art and can be employed to assess the beneficial effects of CVAC programs.

Modulation of stem cell counts in the peripheral blood, prior to and/or following mobilization, is indicative of efficacious CVAC treatments. Similarly, modulation of immune effector cell counts prior to and/or following mobilization is indicative of efficacious CVAC treatment. Modulation of hematocrit is indicative of CVAC efficacy for mobilization of stem cells, engraftment of stem cells, or recovery from stem cell therapy. Conversely, a lack of change in the user's hematocrit (or with any of the physiological markers described herein) does not necessarily indicate that the CVAC treatments are not achieving positive results. Angiogenesis within affected tissues can also be a physiological marker used to assess CVAC efficacy. Modulation of vessel development within the tissues or body of a user during or following one or more CVAC sessions is indicative of efficacious CVAC treatments. Again, by example only, angiogenesis may be assessed by a variety of imaging and detection methods including dyes, MRI, fluoroscopy, endoscopy, and other means known in the art. Additionally, initiation or modulation of VEGF expression within affected tissues during or following one or more CVAC sessions is also indicative of efficacious CVAC treatment. Modulation of EPO production following one or more CVAC sessions is also a physiological marker used to assess the efficacy of CVAC treatments. In one embodiment of the present invention, increases in the expression of EPO indicate efficacious CVAC treatments. Similarly, when blood-gas analysis is the physiological marker used to assess CVAC efficacy, modulation of the dissolved gasses in the blood during or following one or more CVAC sessions is indicative of efficacious CVAC treatment. Typical gasses monitored include oxygen, carbon dioxide, and nitrogen. However, any gas found within the blood may be monitored for assessment of CVAC efficacy. When blood-perfusion of the tissues is the physiological marker used to assess CVAC efficacy, increases in blood volumes or blood exchange and combinations thereof within tissues during or following one or more CVAC sessions are indicative of the efficacious CVAC treatment.

Engraftment and recovery following transplantation can also be assessed utilizing any of the methods detailed above. By way of example, flow cytometry for the determination of Mean Fluorescence Index (MFI) or Mean Reticulocyte Volume (MRV) can be utilized to assess CVAC efficacy related to engraftment following transplantation. Similarly, complete blood counts can be performed to assess recovery following transplantation therapy. Additional criteria for assessing the mobilization of stem cells, engraftment of stem cells, and recovery following stem cell therapy will be known by those of skill in the art and can be employed to assess the beneficial effects of CVAC programs.

A method for prevention, treatment, and/or amelioration of hypertension is disclosed herein. Additionally, a method for improving erythropoiesis is disclosed herein. Furthermore, a method for stem cell mobilization, stem cell engraftment, and modulation of recovery following stem cell therapy by administration of various environmental pressure levels for hypoxic conditioning is disclosed herein. Previously described PVU and CVAC methodology is used to implement the methods for mobilizing stem cells, engrafting stem cells, and recovering from stem cell therapy, and alternative PVUs can be used with the disclosed methodologies. Administration of at least one CVAC session or series of sessions facilitates the treatment, prevention, and/or amelioration of hypertension described herein. Administration of at least one CVAC session or series of sessions facilitates improvement of erythropoiesis. Further, administration of at least one CVAC session or series of sessions facilitates mobilization of stem cells, engraftment of stem cells, and recovery following stem cell therapy.

### EXAMPLE

### Example 1

To assess the efficacy of CVAC sessions, four individuals were administered CVAC sessions and their red blood cell counts hematocrit were subsequently measured and the levels recorded. Increases in red blood cell counts are indicative of CVAC session efficacy, and changes in hematocrit similarly indicate changes in erythropoiesis. For the study, CVAC sessions were administered to a group of four individuals for 40 minutes, 4 times a week, over an 8 week period. Red blood cell levels (RBC) were measured at 5 different intervals during the 8 week test period. The results of the study were as follows:

RBC Mean Increase: 4.7%

The increases in RBC's indicate that CVAC sessions were successful in positively modulating red blood cell counts as well as hematocrit, and both measurements are indicative of increased erythropoiesis. Thus, the administration of CVAC sessions successfully improved erythropoiesis in this 8 week study.

### Example 2

In the same study as example 1, to assess the efficacy of CVAC sessions four individuals were administered CVAC sessions and their hematocrit was subsequently measured and the levels recorded. Changes in hematocrit indicate changes red blood cell concentration as well as indicating changes in erythropoiesis. For the study, CVAC sessions were administered to a group of four individuals for 40 minutes, 4 times a week, over an 8 week period. Hematocrit (HCT) was measured at 5 different intervals during the 8 week test period. The results of the study were as follows:

HCT Mean Increase: 5.3%

The increases in HCT, both alone in combination with the RBC increase as described in example 1, indicate that CVAC sessions were successful in positively modulating hematocrit levels and are further indicative of increased erythropoiesis. Thus, the administration of CVAC sessions successfully improved erythropoiesis in this 8 week study.

### Example 3

To assess the efficacy of CVAC sessions, 13 individuals, all between the ages of 20 and 40 years old, were administered CVAC sessions and changes in their erythropoietin (EPO) levels were measured. Frequency of CVAC administration was for one hour per day, 5 days per week, for seven weeks. Increases in EPO were measured prior to administration of CVAC and three hours post-administration of CVAC, and EPO concentration is expressed as mIU/ml. Thus changes in EPO can be represented by the formula: deltaEPO=Post-CVAC EPO mIU/ml−pre-CVAC EPO mIU/ml. The study found that EPO levels changed significantly over the study period in the population. Specifically, mean changes in EPO concentration increased from 0.2 mIU/ml following the first 2 weeks of CVAC administration to 2.0 mIU/ml following 8 weeks of the CVAC administration. The significant changes in EPO levels found in the study population indicate that the administration of CVAC sessions can positively modulate EPO production, hence providing an alternative and efficacious method to exogenous EPO administration.

### Example 4

Two diabetic subjects (Type-1 and Type-2) were administered 20 minute CVAC sessions, three times a week over a 9 week period. Triglicerides (TGC), Cholesterol levels (HDL and LDL), and Hemoglobin A1c levels were assessed at time points during the study period. Study time periods and results were as follows:
Subject #1: Type-2 diabetic, female

Subject #2: Type-1 diabetic, male

| | Baseline | | 4 Weeks | | 9 Weeks | |
|---|---|---|---|---|---|---|
| Physiological | Subject | Subject | Subject | Subject | Subject | Subject |
| Marker | #1 | #2 | #1 | #2 | #1 | #2 |
| Triglycerides | 102 | 81 | 118 | 85 | 101 | n/d |
| (TGC) | | | | | | |
| HDL | 49 | 72 | 49 | 76 | 49 | n/d |
| LDL | 106 | 111 | 67 | 99 | 84 | n/d |
| HbA1c | 6.7 | 8.4 | 6.8 | 7.6 | 7.1 | n/d |
| (LDL + TGC)/ | 4.2 | 2.7 | 3.8 | 2.4 | 2.1 | n/d |
| HDL | | | | | | |

The results from the two different subjects show a significant drop in their (LDL+TGC)/HDL ratios, indicating improvement in HDL as well as reductions in LDL and/or TGC. Thus in this study, the administration of CVAC sessions resulted in a greater than 9% reduction in the (LDL+TGC)/HDL ratio, successfully reduced the LDL and TGC levels of diabetic individuals, and raised the HDL levels in the diabetic individuals. It may additionally result in at least a 5% reduction in the (LDL+TGC)/HDL ratio, at least a 5-10% reduction in the (LDL+TGC)/HDL ratio, or greater than a 10% reduction in the (LDL+TGC)/HDL ration.

The aspects and embodiments of the present invention described above are only examples and are not limiting in any way. Various changes, modifications or alternations to these embodiments may be made without departing from the spirit of the invention and the scope of the claims.

What is claimed is:

1. A method of treating hypertension in a mammal comprising the step of administering at least one CVAC session, said CVAC session having a start point, an end point and more than one target which is executed between said start point and said end point.

2. The method of claim 1, further comprising the step of measuring efficacy of CVAC sessions via changes in physiological markers.

3. The method of claim 2, wherein the physiological marker measured is selected from among:
blood pressure;
plasma lipid levels;
HIF-1α expression;
VEGF production;
Hematocrit;
Erythropoietin (EPO) production;
angiogenesis within tissues;
blood-perfusion of tissues;
or the oxygenation of tissues in the mammal.

4. The method of claim 1, further comprising the step of administering least one pharmaceutical compound.

5. The method of claim 1, wherein the user can modulate the parameters of a session.

6. A method of modulating red blood cell production in a mammal comprising the step of administering at least one CVAC session, said CVAC session having a start point, an end point and more than one target which is executed between said start point and said end point.

7. The method of claim 6, further comprising a step of extracting blood from the mammal.

8. The method of claim 6, further comprising the step of measuring efficacy of the at least one CVAC session via changes in physiological markers.

9. The method of claim 8, wherein the physiological marker measured is selected from among:
HIF-1α expression;
VEGF production;
Hematocrit;
Erythropoietin (EPO) production;
angiogenesis within tissues;
blood-perfusion of tissues;
or the oxygenation of tissues in the mammal.

10. A method of mobilizing stem cells in a mammal comprising the step of administering at least one CVAC session, said CVAC session having a start point, an end point and more than one target which is executed between said start point and said end point.

11. The method of claim 10, further comprising at least one step of collecting stem cells from the mammal.

12. The method of claim 10, further comprising the step of administering least one pharmaceutical compound.

13. The method of claim 10, further comprising the step of administering at least one growth factor.

14. The method of claim 13, wherein the at least one growth factor is G-CSF.

15. The method of claim 13, wherein the at least one growth factor is a combination of G-CSF and EPO.

16. The method of claim 10 wherein the user can modulate the parameters of a session.

17. A method of facilitating stem cell engraftment in a mammal comprising the step of administering at least one CVAC session, said CVAC session having a start point, an end point and more than one target which is executed between said start point and said end point.

18. The method of claim 17, wherein said at least one CVAC session is administered prior to the administration of the stem cell graft to the mammal.

19. The method of claim 17, wherein said at least one CVAC session is administered following the administration of the stem cell graft to the mammal.

20. The method of claim 17 wherein the user can modulate the parameters of a session.

21. A method of facilitating recovery following administration of a stem cell therapy in a mammal comprising the step of administering at least one CVAC session, said CVAC session having a start point, an end point and more than one target which is executed between said start point and said end point.

22. The method of claim 21, further comprising the step of measuring efficacy of the at least one CVAC session via changes in physiological markers.

23. The method of claim 22, wherein the physiological marker measured is selected from among:
Mean Fluorescence Index (MFI);
Mean Reticulocyte Volume (MRV);
VEGF production;
Hematocrit;
Erythropoietin (EPO) production;
or the oxygenation of tissues in the mammal.

24. The method of claim 21 wherein the user can modulate the parameters of a session.
